(19) United States
(12) Patent Application Publication
(10) Pub. No.: US 2004/0096522 A1
(43) Pub. Date: May 20, 2004

(54) Vitamin/mineral complex

(76) Inventors: Mikhail I Lazarev, Moscow (RU); Rustam K Yenileyev, Moscow (RU)

Correspondence Address: LANIER FORD SHAVER & PAYNE, P O BOX 2087, HUNTSVILLE, AL, 35804, US

(21) Appl. No.: 10/343,781

(22) PCT Filed: Feb. 5, 2002
(86) PCT No.: PCT/RU02/00034

(30) Foreign Application Priority Data
Feb. 14, 2001 (RU) 2001 104090

(51) Int. Cl. A61K 31/355; A61K 33/04

(52) U.S. Cl. 424/702; 514/458

(57) ABSTRACT

The invention regards production of vitamin-mineral systems intended for prophylaxis and treatment. The technical result consists in minimization of the doses of consumption of vitamins and minerals at satisfaction of the needs of human organism, in operability of systems, in decrease of labor input for their production and in augmentation of the terms of their storage. The nature of invention is that vitamin-mineral system is made from three-five preparative forms of various composition intended for separate taking, thus each of such forms contains compatible from the point of view of assimilation and storage vitamins and minerals.

Achievement of announced technical result is also promoted by series of separate attributes.

The invention regards food and pharmaceutical industry: production of vitaminous-mineral systems (complexes) and food additives used for prophylaxis and treatment of vitamin and mineral deficit (hereinafter—deficit).

### Level of Technique

There exists “Complivit” system, that includes 19 components in tablet—vitamins A; E; C; B1; B6; B12; Riboflavinmononucleotide; Nicotinamidum; acid folic; rutin; acid lipoid; calcium pantothenate and minerals: iron (II) sulphate 7-aqueous; copper sulphate; lime phosphate; cobaltous sulphate 7-aqueous; manganese (II) sulphate 5-aqueous; zincum sulphate 7-aqueous; magnesium phosphate disubstituted (composition is specified on packaging of drug which is manufactured by OAO “UFAVITA”). Disadvantage of Complivit is that not all the listed vitamins and minerals are compatible one with another at long-term storage and in assimilation by human organism.

The exists “Duovit” system, consisting of two multi-colored dragees, at separate reception of the dragees during a day diurnal needs of human organism in vitamins and minerals is satisfied. Thus one dragee (red) contains 11 vitamins: A; D3; C; PP; E; B1, B6, B12; calcium pantothenate; folic acid, and another (dark blue)—8 minerals: magnesium; calcium; phosphorus; iron; Zincum; copper; manganese; molybdenum. The mention dragees also include shugar, synthetic colorants and natural aromatic additives (description is on packaging of the drug, manufactured by KRKA).

From widely open sources of information one may find out, that zinc reduces assimilation of copper and competes for assimilation with iron and calcium, and calcium and iron reduce assimilation of manganese owing to what inclusion into one dragee the specified minerals assumes augmentation of their diurnal dosages for satisfaction of mentioned diurnal needs that is a disadvantage of Duovit. Separation of system into two compositions, one of which includes vitamins, and the other—minerals, is not enough.

### Nature of Invention

### Aim of the Invention—Human Health Improvement.

The task of the invention—creation of the system at which separate reception of compositions provides satisfaction of needs of human organism in vitamins and minerals with minimization of taking doses, considering interference of vitamins and minerals.

Technical result of use of the declared invention consists in: a) minimization of daily doses of vitamins and minerals consumption with satisfaction of diurnal needs of organism for them; b) operability of vitamin-mineral systemes including vitamins and minerals incompatible from the point of view of assimilation by organism; c) lowering production costs of vitamin-mineral systems including vitamins and minerals interreacting one with another; d) augmentation of shelf-lifes of vitamin-mineral systems including vitamins and minerals interreacting one with another.

Effect of the announced invention consists in improvement of assimilation by human organism of the components which are included in various compositions of the system, owing creating the conditions for their synergistic interaction and exception—for antagonist. The nature of the invention consists in separation of the system into three-five compositions of fixed mixture expressed below by individual distinguishing characters, determined by age and primary diet of user.

It is proposed the structure of vitamin-mineral system including vitamins and minerals interreacting with one another in the process of manufacturing and/or storage, and/or including vitamins and minerals competing for assimilation by organism, and/or including vitamins and minerals relaxing action of one another in physiological level, containing preparative forms, distinguished by that they are made consisting of three-five preparative forms of various composition intended for separate reception, with the purpose of exception and/or decrease of the above-mentioned interaction, competition and relaxing action.

The announced structure may also be made in such a form for: the above forms are manufactured as dragees or tablets, or capsules; each of the above is colored in a way to distinct it from the color of other forms; it includes blister; the mentioned forms are placed in blister in such a manner for same color series are located in same rows.

It is proposed the composition of vitamin-mineral system including vitamins and minerals interreacting one with another in the process of manufacturing and/or storage, and/or including vitamins and minerals competing for assimilation by organism, and/or including vitamins and minerals relaxing the action of one another at physiological level, distinguished by that it consists of three-five preparative forms of various composition intended for separate reception, with the purpose of exception and/or decrease of the mentioned interactions, competition and relaxing action.

The announced composition also may be such, that: one of its forms includes at least vitamin E and mineral selenium, another—at least vitamins K, D or only vitamin K and mineral calcium whereas one of the remaining—at least minerals iron and copper, and another—minerals zinc and manganese, thus the system may be of various forms; the mentioned above vitamins and minerals are included only in one of the forms of composition; forms contain auxiliary components; as aids the forms contain colorants.

It is proposed the method of manufacturing the vitamin-mineral system including vitamins and minerals interreacting one with another in the process of manufacturing and/or storage, and/or including vitamins and minerals competing for assimilation by organism, and/or vitamins and minerals relaxing action of one another at physiological level, consisting in that different compositions intended for separate reception, for what are mixed the appropriate components, including colorants, in the quantities according to the formula, after that are made the solid preparative forms of different color distinguished by that are prepared three-five compositions in which are mixed synergetic vitamins and minerals reacting and positively influencing one to another, for exception and/or decrease of the mentioned interactions, competition and relaxing action.

It is necessary to point out, that the applicant is aware of combined preparations, for example, estrogen-hestagen ones, being the systems, containing several compositions of different color, into which are included various components (M. D. Mashkovsky “Medicins”, fourteenth edition, vol. 2, M. “Novaya Volna”, 2000, pages 49-55). However, the number of compositions included in these systems and also the materials from which they are made, are determined not by exception of negative interaction between the components at chemical and physiological levels and not by affiliation in one composition of synergetic components, but the necessity of stage-by-stage influence to a human organism depending on its actual state which corresponds the appropriate biochemistry of blood (in particular—hormonal composition and so forth). On the basis of the above at using in known drugs the attributes similar to the declared other result is achieved in comparison with the mentioned above, in this connection implementation of such attributes in the announced system is not apparent for an expert working in the sphere of pharmacology.

In particular, revealing distinguishing characters of the declared structure, compositions and method, the authors, having carried out analysis of open sources of information and having received experimental confirmation of synergistic and antagonical action of components, have analytically came to the conclusion regarding demands to availability of various elements of the system determining the structure of the latter, in other words—regarding the structure of complex composition, determined by the set of materials that was reflected in the specified minimum number of compositions of the system providing the needs for vitamins and minerals of human organism, equal to three. It is easy to show in concrete example. We shall take the minerals necessary for organism—iron, manganese and calcium. It was pointed out above that iron and calcium reduce assimilation of manganese owing to that manganese should included into a composition free of the first two components, and calcium reduces assimilation of iron (see Griffit V., Vitamins, Herbs, minerals and food additives, M., Publishing house “Grand”, 2000), hence the latters should also be separated. In the result we see that the system should consist of three compositions at least.

Including copper and magnesium into the system, in view of the facts described in “Level of technique” and that iron excess results in deficiency of copper, and excess of manganese in deficiency of magnesium (see A. V. Skal'nyj, Human Microelemetoses, M., “Nauchny Mir”, 1999) assumes further diving the system into 4, 5 and more compositions, however, the practical tests, carried out by the authors, are showing that using more than 5 compositions essentially complicates the sequence and regularity of taking of the system, limiting the range of its application in clinical practice. So the limit—5 compositions of the system—has appeared.

Basing on the mentioned analysis and experiments the authors have revealed the attributes concerning all the set of vitamins and minerals, taking place in fixed modular state and amounting various compositions of the declared system—material, from which the elements of system are made; and in the mentioned practice—concerning the ingredient composition and utilitarian indexes of the compositions, attributes of the method of their receiving and also the form of performance, relative positioning and characteristics of elements of the system.

Information Confirming Possibility of Exercising the Invention

The declared system is intended for taking at the established term, for example, within the day period.

The declared system contains vitamins and minerals in the kind of three-five compositions intended for separate taking of incompatible components, for example, separately by time.

In particular, it may contain 23 components, divided into 3 compositions, which are intended for taking within a day. In this case components in the composition may be separated in such a manner that one of them contains vitamins B1, B3, B6, and also minerals—iron, copper, molybdenum and iodine, another one—vitamins K, H, B2, B5, B6, B9, B12, D3 and minerals—chrome and calcium and the last one—vitamins A, E, C and minerals—magnesium, manganese, selenium, zinc.

Compositions of vitamins and minerals may be obtained by mechanical blending the components in the required quantities according to the formula.

Calcium stearate (2%) and aerosil (3%) or follow-up water (5%) and solution of gelatine (1%) may be added to the obtained admixtures.

At reception of the mentioned firm forms aids may be utilized: food gelatine; nipagin; stearinic acid; sugar; potato starch; talc; calcium stearate; aerosil. As aids also may be utilised food colorants, for example: E128; Red 2G;

E-129.a; Red charming AC; E-131; Dark blue patented V; E-133; Dark blue brilliant FCF, etc.

Thus each composition may be colored to distinct from the others, and they may be packed in blister in such a way for in one series firm forms of one color are located.

Received material may be pressed or granulated for receiving firm preparative forms (dragee, tablets and so forth), and also, for example, for dosing and packaging in capsules.

As it was already pointed out in art. “Nature of invention”, on the reasons specified there the system may consist of four or five compositions. So, the first composition may include vitamins B1, B3, B6 and minerals Fe, Mo, the second—minerals Cu, J, the third—B2, B5, B6, B9, B12, D3, H, K and minerals Ca, Cr, the fourth—vitamins A, B, C and minerals Mg, Mn, Se, Zn.

In the case with five compositions the system may be made as follows. The first composition—vitamins B1, B3, B6 and minerals Fe, Mo, the second—minerals Cu, J, the third—B2, B9, B12, D3, K and minerals Ca, P, Mg, Zn, the fourth—vitamins B5, B6, H and mineral Cr, and the fifth—vitamins A, E, C and minerals Mn, Se.

Use of the declared invention allows to achieve the technical result expressing in the following.

a) In minimization of doses of reception of vitamins and minerals with satisfying the needs of organism. Really, separation on various preparative forms of incompatible from the point of view of assimilation by organism vitamins and minerals at decrease of their total taking allows to raise their assimilation by organism.

At the same time it is known, that vitamin E and selenium, and also vitamin K and calcium react synergetic; vitamin D is necessary for assimilation of calcium; copper promotes assimilation of iron, etc. Inclusion of components positively influencing one to another into one composition also promotes achievement of the specified result.

The mentioned separation will allow to decrease diurnal dosage of various vitamins and mineral substances in 1,5. 2,5 times. Apparently, optimization of integral consumption of vitamins and minerals (in particular, decrease of accepted doses of some vitamins and minerals) will promote improvement of health of people, that will especially prove among the persons, suffering the diseases connected with disbolism.

b) Operability of vitamin-mineral systems including incompatible by organism vitamins and minerals from the point of view of assimilation.

Coloring of preparative forms in various colors facilitates correct taking of drug.

The mentioned placement of variously colored compositions in one blister facilitates observing the sequence of taking the compositions of the system (taking of mentioned preparative forms foreseen destruction of blister owing to what dragee or tablet or capsule is taken out).

c) Decreasing labor input at production of vitamin-mineral systems including vitamins and minerals interreacting one with another—disappears the necessity of implementation of auxiliary additives providing stability of compositions in traditional systems including such vitamins and minerals. In conditions of large quantity production decrease of the mentioned labor input is expected 5-10%.

c) Increasing in 1-2-3 times the shelf-lives of vitamin-mineral systems including vitamins and minerals interreacting one with another which takes place naturally, due to absence of such vitamins and minerals in various compositions of the system.

The submitted quantitative assessments are obtained by the applicant at carrying out initiative researches and based on results of biochemical analysis.

### The Formula of Inventions

1. Structure of vitamin-mineral system including vitamins and minerals, interreacting one with another in the process of manufacturing and/or storage, and/or including vitamins and minerals competing for assimilation by human organism, and/or including vitamins and minerals relaxing action of one another at physiological level, containing preparative forms, distinguished by that it consists of three-five preparative forms of various compositions intended for separate taking, for exception and/or decrease of the mentioned interaction, a competition and relaxing action.

2. Structure under item 1, distinguished by that the mentioned forms are made as dragees or tablets or capsules.

3. Structure under item 2, distinguished by that each of the mentioned forms is colored so that to differ from the color of other forms.

4. Structure of item 2, distinguished by that it includes blister.

5. Structure under item 4, distinguished by that the mentioned forms are placed in blister in such a manner that in one series are the forms of one color.

6. Composition of vitamin-mineral system including vitamins and minerals, interreacting one with another in the process of manufacturing and/or storage, and/or including vitamins and minerals competing for assimilation by human organism, and/or including vitamins and minerals relaxing the action of one another at physiological level, distinguishing by that it consists of three-five preparative forms of various composition intended for separate taking, for exception and/or decrease of the mentioned interactions, competitions and relaxing action.

7. Composition according to item 6, distinguished by that one of the forms includes at least vitamin E and mineral selenium.

8. Composition according to item 7, distinguished by that the other form includes at least, vitamins K, D and mineral calcium.

9. Composition according to item 7, distinguished by that the other form includes least vitamin K and mineral calcium.

10. Composition according to items 8 or 9, distinguished by that one of the remaining forms includes at least minerals iron and copper.

11. Composition according to item 10 distinguished by that another of the remaining forms includes at least minerals zinc and manganese.

12. Composition under items 7-11, distinguished by that the mentioned vitamins and minerals are included only into one of the forms.

13. Composition under item 6, distinguished by that the forms contain aids.

14. Composition under item 13, distinguished by that the forms contain colorants as aids.

15. Method of manufacturing vitamin-mineral system including vitamins and minerals, inter-reacting one with another in the process of manufacturing and/or storage, and/or including vitamins and minerals competing for assimilation by organism, and/or including vitamins and minerals relaxing the action of one another at physiological level, consisting in the fact that different compositions intended for separate taking are prepared, for which are mixed the components, including colorants, in the quantities according to the formula, after that the firm preparative forms of different color are made distinguished by the fact that three five compositions are made by mixing synergetically acting vitamins and/or minerals positively influencing one to another, for exception and/or decrease of the mentioned interactions, competitions and relaxing action.

I claim:

1. A vitamin-mineral composition, comprising:
a) three to five separate dose formulations;
b) each said separate dose formulation having vitamins and minerals;
c) said vitamins and minerals being combined in each of said dose formulation so that each of said formulations includes synergistic vitamins and minerals; and
d) said separate dose formulations being administered to a user at separate times.

2. The composition of claim 1 wherein said dose formulations are made as liquids, capsules, tablets, or lozenges.

3. The composition of claim 2 wherein said dose formulations are colored so that each said separate dose formulation is identified differently from the other separate dose formulations.

4. The composition of claim 2 wherein said dose formulations are contained in a blister pack.

5. The composition of claim 4 wherein said dose formulations in said blister pack are separated in groups by coloring.

6. The composition of claim 1 wherein a first of said separate dose formulations contains at least vitamin E and selenium.

7. The composition of claim 6 wherein a second of said separate dose formulations contains at least vitamins K and D and calcium.

8. The composition of claim 6 wherein a second of said separate dose formulations contains at least vitamin K and calcium.

9. The composition of claim 7 wherein a third of said separate dose formulations contains at least iron and copper.

10. The composition of claim 8 wherein a third of said separate dose formulations contains at least iron and copper.

11. The composition of claim 9 wherein a fourth of said separate dose formulations contains at least zinc and manganese.

12. The composition of claim 10 wherein a fourth of said separate dose formulation contains at least zinc and manganese.

13. The composition of claim 1 wherein vitamins E, D, K and minerals selenium, calcium, iron, copper, zinc, or manganese are contained in only one of said separate dose formulations.

14. The composition of claim 1 wherein said separate dose formulations contain excipients or coatings.

15. The composition of claim 14 wherein said excipients or coatings produce color.

16. The composition of claim 1 wherein said vitamins and minerals are combined in each said separate dose formulations so as to minimize negative interactions among said vitamins and minerals.

17. The composition of claim 1 wherein a first of said separate dose formulations contains vitamin B1, a second of said separate dose formulations contains vitamin B2, and a third of said separate dose formulations contains vitamin A.

18. The composition of claim 17 wherein said first of said separate dose formulations contains vitamin B3, said second of said separate dose formulations contains vitamin B5, and said third of said separate dose formulations contains vitamin E.

19. The composition of claim 18 wherein said first of said separate dose formulations contains vitamin B6, said second of said separate dose formulations contains vitamin B6, and said third of said separate dose formulations contains vitamin C.

20. The composition of claim 19 wherein said first of said separate dose formulations contains iron, said second of said separate dose formulations contains vitamin B9, and said third of said separate dose formulations contains magnesium.

21. The composition of claim 20 wherein said first of said separate dose formulations contains iodine, said second of said separate dose formulations contains vitamin B12, and said third of said separate dose formulations contains manganese.

22. The composition of claim 21 wherein said first of said separate dose formulations contains copper, said second of said separate dose formulations contains vitamin D₃, and said third of said separate dose formulations contains selenium.

23. The composition of claim 22 wherein said first of said separate dose formulations contains molybdenum, said second of said separate dose formulations contains vitamin K, and said third of said separate dose formulations contains zinc.

24. The composition of claim 23 wherein said second of said separate dose formulations contains biotin.

25. The composition of claim 24 wherein said second of said separate dose formulations contains calcium.

26. The composition of claim 25 wherein said second of said separate dose formulations contains chromium.

27. The composition of claim 17 wherein the amounts of said vitamins and minerals are about B₁ 1.5 mg, B₃ 20 mg, B₆ 2 mg, B₂ 1.7 mg, B₅ 5 mg, B₉ 200 μg, B₁₂ 3 μg, D₃ 100 IU, biotin 30 μg, K₁ 25 μg, A 3333 IU, E 10 IU, C 80 mg, iron 18 mg, iodine 150 μg, copper 2 mg, molybdenum 250 μg, calcium 100 mg, chromium 25 μg, magnesium 40 mg, manganese 2.5 mg, selenium 25 μg, and zinc 15 mg.

28. A vitamin-mineral composition, comprising:
a) three or more separate dose formulations;
b) each said separate dose formulation having vitamins and minerals;
c) said vitamins and minerals being combined in each of said dose formulation so as to maximize the positive interactions and minimize the negative interactions among said vitamins and minerals; and
d) said separate doses being administered to a user at separate times to avoid the negative interactions among said vitamins and minerals.

29. The composition of claim 28 wherein said dose formulations are made as liquid drops, capsules, tablets, or lozenges, or a combination thereof.

30. The composition of claim 29 wherein said dose formulations are coded so that each said separate dose formulation is identified differently from the other separate dose formulations.

31. The composition of claim 28 wherein said dose formulations are contained in a blister pack.

32. The composition of claim 31 wherein said dose formulations in said blister pack are separated in groups by said coding.

33. The composition of claim 28 wherein a first of said separate dose formulations contains at least vitamin E and selenium.

34. The composition of claim 33 wherein a second of said separate dose formulations contains at least vitamins K and D and calcium.

35. The composition of claim 33 wherein a second of said separate dose formulations contains at least vitamin K and calcium.

36. The composition of claim 34 wherein a third of said separate dose formulations contains at least iron and copper.

37. The composition of claim 35 wherein a third of said separate dose formulations contains at least iron and copper.

38. The composition of claim 36 wherein a fourth of said separate dose formulations contains at least zinc and manganese.

39. The composition of claim 37 wherein a fourth of said separate dose formulation contains at least zinc and manganese.

40. The composition of claim 28 wherein vitamins E, D, K and minerals selenium, calcium, iron, copper, zinc, or manganese are contained in only one of said separate dose formulations.

41. The composition of claim 28 wherein said separate dose formulations contain excipients, coatings, or a combination thereof.

42. The composition of claim 41 wherein said excipients, or coatings, or the combination thereof, produce color.

43. A method of delivering vitamin-mineral compositions, comprising the steps of:
a) combining vitamins and minerals to promote synergistic activity in at least three dose formulations;
b) separating vitamins and minerals to avoid negative interactions in said at least three dose formulations; and
c) consuming said at least three dose formulations at different times to avoid said negative interactions.

44. The method of claim 43 further comprising the step of coding said dose formulations to indicate the time at which said dose formulation should be consumed.

45. The method of claim 44 further comprising the step of placing said dose formulations in a blister pack.

46. The method of claim 45 wherein the step of combining includes combining up to 13 vitamins and 10 minerals.
